(19) United States
(12) Patent Application Publication
(10) Pub. No.: US 2008/0171009 A1
(43) Pub. Date: Jul. 17, 2008

(54) KERATIN FIBRE MAKEUP KIT

(75) Inventors: Frederic AUGUSTE, Chevilly-Larue (FR); Jean-Yves Fouron, Bourg-La-Reine (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/969,286

(22) Filed: Jan. 4, 2008

Related U.S. Application Data
(60) Provisional application No. 60/885,637, filed on Jan. 19, 2007.

(30) Foreign Application Priority Data
Jan. 4, 2007 (FR) 07 52524

(51) Int. Cl. A61K 8/00

(52) U.S. Cl. 424/70.7; 424/63

(57) ABSTRACT

Keratin fibre makeup kit containing at least:
a first composition with a surface energy of less than or equal to 30 mN/m, and
a second composition with a surface energy such that the difference between its surface energy and that of the first composition is greater than or equal to 5 mN/m, and a viscosity of between 0.001 and 2 Pa·s.

This non provisional application claims the benefit of French Application No. 07 52524 filed on Jan. 4, 2007, and U.S. Provisional Application No. 60/885,637 filed on Jan. 19, 2007, both incorporated herein by reference.

The present invention relates to a keratin fibre makeup kit and to the corresponding makeup process.

The present invention relates to making up keratin fibres, for instance the eyelashes, the eyebrows and the hair, and more particularly to making up the eyelashes.

The makeup kit according to the invention may be in the form of a makeup kit for the eyelashes or the eyebrows, or a hair makeup kit. More especially, the invention relates to a mascara. It may especially be an eyelash makeup kit.

There are in practice essentially two types of mascara formulation, namely, on the one hand, mascaras with an aqueous continuous phase, known as “emulsion mascaras”, which are in the form of an emulsion of waxes in water, and, on the other hand, mascaras with a solvent or oil continuous phase, which are anhydrous or have a low content of water and/or water-soluble solvents, known as “waterproof mascaras”, formulated in the form of a dispersion of waxes in non-aqueous solvents. Moreover, certain mascaras are in the form of emulsions, of wax in water, which are also termed “waterproof”. These mascara compositions are characterized by the presence of at least one latex or pseudolatex, namely a colloidal suspension of a film-forming polymer, which imparts water resistance.

Conventionally, mascaras comprise at least one film-forming polymer that especially enables coating of the eyelashes.

One desired aim of the present invention is that of providing a single device for offering mascara users a means for producing varied makeup effects, which may be readily modified depending on the manner in which the application is performed.

Document EP 953 332 discloses a makeup composition comprising at least one aqueous dispersion of particles of film-forming polymer and at least one thickener, wherein said composition has a viscosity, measured at a shear of 500 s⁻¹, ranging for example from 0.001 Pa·s to 2 Pa·s, and also an associated keratin fibre makeup process allowing the formation of pearls distributed along the keratin fibres, following an application at a suitable rate.

Compositions for obtaining, after application to keratin fibres, drops, which are preferably transparent, characterized by the presence of a particular polymer, are moreover disclosed in document FR 2 844 706.

The inventors have found that it is possible to obtain a wide range for example of types of keratin fibre makeup by successive application of two compositions defined, respectively, for the first of them, by a maximum surface energy of 30 mN/m, and, for the second of them, by a surface energy exhibiting a value difference in surface energy greater than or equal to 5 mN/m relative to the surface energy of the first composition, and a viscosity of between 0.001 and 2 Pa·s.

Nonlimiting examples of makeup results are represented diagrammatically in the attached figures.

### BRIEF DESCRIPTION OF THE DRAWINGS

FIG. 1 represents a portion of eyelash fringes made up according to one of the alternatives of the makeup process in accordance with the present invention,

FIG. 2 represents a portion of eyelash fringes made up according to another alternative of the makeup process in accordance with the present invention,

FIG. 3 represents a portion of eyelash fringes made up according to yet another alternative of the makeup process in accordance with the present invention.

In all the figures, a portion of eyelid 1 is represented.

In other words, the implementation of the makeup kit according to the present invention makes it possible to modify not only the amount, but also the location on the keratin fibres, of the desired deposit of material along said keratin fibres. While not bound by theory, it is believed, indeed, that the characteristics of the first and second compositions give rise to the displacement of material by capillary action, which, after drying, and thus hardening, set the makeup result in the form, for example, of single or multiple droplets on each eyelash taken separately, or alternatively in the form of “bridges” between the keratin fibres. There is thus formation of an “excess of material”, which may or may not have the same optical effect and especially the same colour as that of the first composition, which affords a particular makeup result when compared with that obtained with common mascaras, which, on the contrary, are generally directed towards uniform makeup along each keratin fibre.

The makeup process according to the present invention may also be used for making up the eyelashes so as to emphasize the look by virtue of giving an impression of a large deposited volume. In particular, the large deposit of material located at a precise point along the eyelashes, obtained by means of the implementation of the process according to the present invention, makes it possible to achieve this impression.

Thus, according to a first aspect, the present invention relates to a keratin fibre makeup kit comprising at least:
a first composition with a surface energy of less than or equal to 30 mN/m, and
a second composition with a surface energy, before drying, such that the difference between this (its) surface energy and that of the first composition is greater than or equal to 5 mN/m, and a viscosity of between 0.001 and 2 Pa·s.

According to a second aspect, the present invention relates to a process for making up and/or for non-therapeutically caring for keratin fibres, wherein it comprises at least one step of applying a first composition in accordance with the present invention to all or part of the keratin fibres, forming a first deposit, and at least one step of applying a second composition in accordance with the present invention over all or part of the first deposit and/or of the keratin fibres.

In the context of the present invention, the term “keratin fibres” especially means the hair, the eyelashes and the eyebrows.

The makeup kits in accordance with the invention comprise a physiologically acceptable medium, especially a cosmetically acceptable medium, i.e. a medium that is compatible in particular with keratin fibres such as the hair, the eyelashes and the eyebrows.

In the context of the present invention, the term “cosmetically acceptable” refers to a compound whose use is compatible with application to keratin materials.

The manifestation of the makeup result desired in the context of the present invention is especially observed when the second composition is successively deposited after the first composition. Thus, at least two application actions are necessary to obtain the desired makeup effect.

The application of the two compositions may be performed using any application means, or applicator, as is described hereinbelow.

The first and the second compositions may also be respectively termed the “base coat” and the “top coat”.

In the description hereinbelow, the term “dry deposit” means the deposit formed after applying and drying a composition on a support, especially after drying this deposit for 24 hours at room temperature (23±2° C.), and a controlled relative humidity RH of 50±5%.

More particularly, the present invention relates to a keratin fibre makeup kit comprising at least:
a first composition with a surface energy, measured on the dry deposit, of less than or equal to 30 mN/m, and
second composition with a surface energy, measured before drying, such that the difference between this surface energy and that of the first composition, measured on the dry deposit, is greater than or equal to 5 mN/m, and a viscosity of between 0.001 and 2 Pa·s.

Measurement of the Surface Energy

The surface energy may be measured in various ways.

For the first composition, the surface energy is measured, for example, on the dry deposit. The measurement is then performed in the following manner:

Deposits of the first composition with a wet thickness of 100 μm are produced on the Bakelite sample holder of a contact-angle tensiometer such as the DAT 1100 tensiometer sold by the company Fibro (Sweden).

The deposit of the first composition is produced by laying flat on the sample holder of the tensiometer a rectangular metal stencil of dimensions 10×20 mm and 100 μm thick. The interior of the stencil frame is filled with the composition and the surface is leveled off using a standard microscope slide (glass slide, dimensions 26×76 mm), in order to remove the excess material.

After drying these deposits for 24 hours at room temperature (23±2° C.) and a controlled relative humidity RH of 50±5%, the contact angle measurements are taken at 20±1° C. and 50% relative humidity with water using at least three of the following solvents: water, glycerol, parleam oil, diiodomethane and formamide.

For each of the solvents, a drop of solvent, with a volume of between 2 and 10 microlitres, is deposited on the dry deposit formed by the first composition. The optical and image-analysis systems of the tensiometer make it possible to follow the change over time of the spreading of the drop. The contact angle of the liquid on the dry deposit formed by the first composition corresponds to the angle between the composition/liquid contact surface and the tangent to the drop passing through the point of encounter of the drop, the air and the surface. Two angles are measured, one on each side of the drop, and the computer calculates the average of these two angles.

The contact angle value used to determine the critical surface energy is the mean of the contact angles measured for 5 to 10 drops on the deposit formed by the first composition, the angle values being taken after a contact time between the drop of solvent and the deposit formed by the first composition equal to 1 second.

To calculate the surface energy of each first composition, Young's model is used, with the Fowkes equations (cf. “Determination of interfacial tensions, contact angles, and dispersion forces in surfaces by assuming additivity of intermolecular interactions in surfaces” Fowkes, F. M., Journal of Physical Chemistry (1962), 66, 382 and “Contact angle, wet ability and adhesion, advances in chemistry series”, No 43 Fowkes (1964)).

Knowing the dispersive and polar components of the solvents (respectively, γ_{L}^{d} and γ_{L}^{p} expressed in mJ/m² also with the superficial energy γL defined by γ_{L}=γ₁^{d}+γ₁^{p}) and the contact angle θ measured between the solvent and the deposit of composition, a graph

$$\frac{γ L ( 1 + cos θ )}{2 \sqrt{{γ}_{L}^{d}}}$$

is plotted as a function of \sqrt{(γ_{L}^{p}/γ_{L}^{d})}.

The shape of this graph approaches a straight line of slightly positive slope: the slope of this straight line corresponds to \sqrt{(γ_{S}^{p})} and the ordinate to the origin to \sqrt{(γ_{S}^{d})}. The values of γ_{S}^{d} and γ_{S}^{p} are thus obtained, i.e. the dispersive and polar components of the free surface energy of the deposit formed by the first composition.

The surface energy of the composition γ_{S} is defined by the relationship: γ_{S}=γ_{S}^{d}+γ_{S}^{p}.

For the second composition, the surface energy is measured before drying the composition, in other words on the formula in liquid form, according to the Wilhelmy slide method described in standard ISO 304-1985 (F), using a platinum measuring body as mentioned in section 5.2 a). The measurement is performed with a Krüss K12 tensiometer at a temperature of 20° C., the depth of immersion of the slide into the composition is set at 2 mm, and the other parameters are chosen by the machine software so as to avoid failure of the interfacial film.

In the case of liquids, the surface energy is also known as the surface tension. The cleaning of the slide and of the glassware for the tensiometer is detailed hereinbelow for this protocol.

After cleaning with a suitable solvent (for example acetone), the slide is treated with a Bunsen burner flame until it reddens. As regards the glassware (crystallizing dish), it is immersed in a solution of detergent of Decon type for 12 hours. It is then rinsed several times with ultrapure water and dried with a heat stripper.

The quality of the cleaning is checked by measuring the surface tension of ultrapure water. 72.3±0.5 mN/n should be obtained.

Measurement of the Viscosity

The viscosity is measured at 25° C.±0.5° C. using a Haake RS600 imposed-stress rheometer from the company Thermo Rheo, equipped with a spindle of cone/plate geometry with a diameter of between 2 cm and 6 cm and a contact angle of between 1° and 2°, the choice of the spindle being made as a function of the viscosity to be measured (the more liquid the formula, the greater the diameter of the chosen cone and the smaller the angle). The measurement is taken by imposing on the oil sample a logarithmic ramp of shear gradient γ′ ranging for example from 10⁻³ s⁻¹ to 1000 s⁻¹, for a duration of 5 minutes. The rheogram representing the change in viscosity as a function of the shear gradient γ is then plotted. The value retained is the value of the viscosity at 500 s⁻¹, whether it is measured at this gradient or extrapolated from the plot if no experimental point corresponds to this value.

First Composition

The first composition has a surface energy of less than or equal to 30 mN/m.

According to one preferred embodiment, the surface energy of the first composition is less than or equal to 25 mN/m and even more preferably less than 20 mN/m.

In order to achieve such a surface energy, the first composition advantageously comprises silicone and/or fluoro compounds, chosen especially from silicone waxes and/or fluoro waxes, pigments with a silicone and/or fluoro surface coating or treatment, and mixtures thereof.

The composition may especially comprise fluoro waxes and/or silicone waxes or waxes comprising silicone and/or fluoro parts.

As regards the general definition of waxes, reference will be made to the paragraph devoted thereto in the description hereinbelow.

Fluoro Waxes

The term “fluoro wax” means a compound comprising a carbon backbone and a number of fluorine atoms that is preferably greater than that of the hydrogen atoms, the carbon backbone possibly being interrupted with at least one oxygen atom. This wax comprises only fluorine, hydrogen and carbon and possibly oxygen atoms.

Among the fluoro waxes with a carbon backbone, mention may be made especially of the fluoro esters corresponding to formula (VI) below:

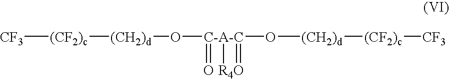

in which:

R₄ represents a hydrogen atom or the radical

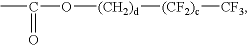

A represents an alkylene or alkenylene hydrocarbon-based chain, said chain being linear or branched, optionally hydroxylated, and containing from 1 to 18 carbon atoms,

c range for example s from 1 to 17, and

d range for example s from 1 to 18.

Among the compounds of formula (VI), mention may be made especially of 2F-octylethyl 1,12-dodecanedioate having the following formula:

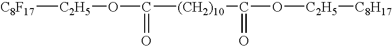

and C₁-C₆ perfluoroalkyl tricitrates, more particularly the perfluorobutyl tricitrate sold under the name Zonyl TBC® by the company DuPont. As other fluoro waxes that may be used in the invention, mention may be made of polytetrafluoroethylene (PTFE) waxes.

Preferably, waxes with a melting point ranging for example from 45° C. to 150° C., especially such as the fluoro esters of formula (VI), are used.

It is also possible to use microdispersions of fluoro waxes, for instance Microdispersion 411 from MicroPowders, which is a mixture of polyethylene waxes, and the product Aquapolyfluo 411 from MicroPowders, which is a mixture of polyethylene wax and of polytetrafluoroethylene.

Silicone Waxes

The silicone waxes that may be used in the composition according to the invention are advantageously substituted polysiloxanes, preferably with a low melting point. They are especially substituted linear polysiloxanes constituted essentially (apart from the end groups) of units of formulae (II) and (III), in the respective molar proportions m and n:

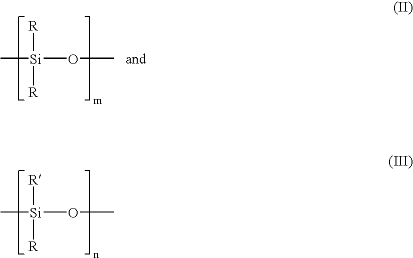

in which:
each substituent R is defined as above,
each R′ independently represents a linear or branched, saturated or unsaturated hydrocarbon-based chain containing from 6 to 30 carbon atoms, or a group —X—R″, each X independently representing:
—O—,
—(CH₂)ₐ—O—CO—,
—(CH₂)_{b}—O—CO—,

a and b independently represent numbers possibly ranging for example from 0 to 6, and each R″ independently represents a saturated or unsaturated hydrocarbon-based chain containing from 6 to 30 carbon atoms,
m is a number possibly ranging for example from 0 to 400 and in particular from 0 to 100,
n is a number possibly ranging for example from 1 to 200 and in particular from 1 to 100,

the sum (m+n) being less than 400 and in particular less than or equal to 100.

These silicone waxes are known or may be prepared according to the known methods. Among the commercial silicone waxes of this type, mention may be made especially of those sold under the names Abil Wax 9800, 9801 or 9810 (Goldschimidt), KF910 and KF7002 (Shin-Etsu) or 176-1118-3 and 176-11481 (General Electric).

The silicone waxes that may be used may also be chosen from the compounds of formula (IV):

R₁—Si(CH₃)₂—O—[Si(R)₂—O]_{z}—Si(CH₃)₂—_{R2} (IV)

in which:

R is defined as above,

R₁ represents an alkyl group containing from 1 to 30 carbon atoms, an alkoxy group containing from 6 to 30 carbon atoms or a group of formula:

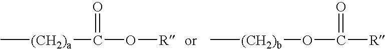

R₂ represents an alkyl group containing from 6 to 30 carbon atoms, an alkoxy group containing from 6 to 30 carbon atoms or a group of formula:

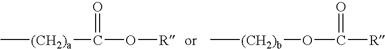

a and b representing a number from 0 to 6,

R″ being an alkyl containing from 6 to 30 carbon atoms,

and z is a number possibly ranging for example from 1 to 100.

Among the silicone waxes of formula (IV), mention will be made especially of alkoxy dimethicones, such as the following commercial products: Abil Wax 2428, 2434 and 2440 (Goldschmidt) or VP 1622 and VP 1621 (Wacker), and also (C₂₀-C₆₀) alkyl dimethicones, in particular (C₃₀-C₄₅) alkyl dimethicones, for instance the silicone wax sold under the name SF-1642 by the company GE-Bayer Silicones.

It is also possible to use hydrocarbon-based waxes modified with silicone or fluoro groups, for instance: siliconyl candelilla, siliconyl beeswax and Flurobeeswax from Koster Keunen.

The fluoro waxes and/or silicone waxes may be present in the composition in a content ranging for example from 0.1% to 50% by weight, preferably ranging for example from 1% to 40% by weight and preferentially ranging for example from 5% to 25% by weight relative to the total weight of the composition.

Pigments with a Silicone and/or Fluoro Surface Coating or Treatment

Still with the aim of achieving the required surface energy, the first composition may comprise pigments treated with a fluoro and/or silicone treatment.

More particularly, the pigments may be coated with all organofluorine compound or a silicone compound. They may comprise a mixture of said pigments treated with a fluoro and/or silicone treatment.

The organofluorine compound coating the pigment may especially be a perfluoroalkyl phosphate or a polyhexafluoropropylene oxide.

Pigments coated with perfluoroalkyl phosphate are described especially in documents U.S. Pat. No. 3,632,744, JP-A-04 330 007 and JP-A-2001-316 223.

The perfluoroalkyl phosphate may be chosen especially from those of formula (1):

[CₘF₂ₘ₊₁CₙH₂ₙO]_{y}PO(OM)_{3-y}

with m being an integer ranging for example from 1 to 21; n an integer from 1 to 14; y=1, 2 or 3; M is —H, an alkali metal cation, an ammonium group or an ammonium group substituted with one to three C₁-C₈ alkyl radicals.

As pigments coated with perfluoroalkyl phosphate, mention may be made of the Covafluor family from LCW-Wackherr and PF 5 Black BL 100 from Daito Kasei Kogyo.

According to another embodiment, the pigment may be coated with a silicone compound, for instance methicones, dimethicones, polyorganosiloxanes comprising perfluoroalkyl perfluoropolyether groups, and perfluoroalkylsilanes.

As coated pigments, the following may be used for example in the context of the present invention:
the pigments coated with perfluoroalkyl phosphate and with butyl acrylate/perfluoro(C₆-C₁₄)alkylethyl acrylate/mercaptopropyl dimethicone copolymer sold under the names Novatech NFP Double Treated Talc, Novatech NFP Double NFP Treated Sericite, Novatech NFP Double Treated Yellow Iron Oxide, Novatech NFP Double Treated Red Iron Oxide, Novatech NFP Double Treated Black Iron Oxide and Novatech NFP Double Treated Titanium Dioxide by the company Daikin;
the pigments coated with perfluoroalkyl phosphate, with butyl acrylate/perfluoro(C₆-C₁₄)alkylethyl acrylate/mercaptopropyl dimethicone copolymer and with hydroxyethyl methacrylate/perfluoro(C₆-C₁₄)alkylethyl acrylate copolymer sold under the names Novatech NFP Triple Treated Talc, Novatech NFP Triple NFP Treated Sericite, Novatech NFP Triple Treated Yellow Iron Oxide, Novatech NFP Triple Treated Red Iron Oxide, Novatech NFP Triple Treated Black Iron Oxide and Novatech NFP Triple Treated Titanium Dioxide by the company Daikin.

The organofluorine compound or the silicone compound coating the pigment may be present in a content ranging for example from 0.01% to 60% by weight, preferably ranging for example from 0.05% to 40% by weight and preferentially ranging for example from 0.1% to 40% by weight, relative to the total weight of the coated pigment.

The coated pigment as described above may be present in the first composition according to the invention in a content ranging for example from 0.01% to 30% by weight, preferably ranging for example from 0.1% to 20% by weight and preferentially ranging for example from 0.5% to 10% by weight relative to the total weight of the composition.

As a component for achieving the required surface energy, the first composition may comprise only fluoro waxes and/or silicone waxes or alternatively only pigments treated with a fluoro and/or silicone treatment or alternatively a mixture of fluoro waxes and/or silicone waxes and of pigments treated with a fluoro and/or silicone treatment.

Oils

Advantageously, the first composition has an oily continuous phase. The oils or organic solvents envisaged in the context of the present invention are detailed hereinbelow.

The expression “composition with an oily continuous phase” means that the composition has a conductivity, measured at 25° C., of less than 23 μS/cm (microSiemens/cm), the conductivity being measured, for example using an MPC227 conductimeter from Mettler Toledo and an Inlab730 conductivity measuring cell. The measuring cell is immersed in the composition, so as to remove the air bubbles liable to form between the two electrodes of the cell. The conductivity reading is taken once the conductimeter value has stabilized. An average is determined on at least three successive measurements.

The total oil content in the first composition may range for example from 30% to 90% by weight, preferably from 40% to 80% by weight and better still from 50% to 75% by weight relative to the total weight of the first composition.

According to another particular embodiment, the first composition comprises at least one volatile oil as defined later.

According to one particular embodiment, when the first composition comprises at least one non-volatile oil, it then exhibits a volatile oil content greater than the content of non-volatile oil. Thus, the content of volatile oil is advantageously between 50% and 100% (limits inclusive), especially between 60% and 100% and preferentially between 80% and 100% by weight relative to the total weight of oil in the first composition.

According to another embodiment, the first composition is anhydrous. The term “anhydrous” means a composition with a content of water and or of water-soluble solvent of less than or equal to 5%, preferably less than or equal to 3% and better still free of added water.

Structuring Agent

The first composition may comprise at least one additional structuring agent for the oil phase, chosen from additional waxes other than the fluoro waxes and/or silicone waxes mentioned above, semi-crystalline polymers and lipophilic gelling agents, and mixtures thereof. Said structuring agents are detailed in the paragraph hereinbelow.

Preferably, the additional structuring agent is present in the first composition in a content ranging for example from 0.1% to 20% by weight and especially from 0.5% to 10% by weight, for example from 1% to 10% by weight, relative to the total weight of the first composition.

Film-Forming Polymer

The first composition may also contain a film-forming polymer. Such a film-forming polymer may be present in the first composition in a solid matter content ranging for example from 0.1% to 20% by weight, especially from 0.5% to 15% by weight and preferably from 1% to 10% by weight relative to the total weight of the first composition.

In general, the first composition may comprise any compound conventionally included in a cosmetic composition for making up keratin fibres, and especially a mascara, provided that its addition in particular respects the required energy surface characteristic.

According to one advantageous embodiment of the invention, the first composition should not act as a solvent for the second composition. There is then said to be incompatibility between the first and the second composition.

The result of this characteristic was rendered by implementing a protocol directed towards measuring the capacity of the second composition, when this second composition has an aqueous continuous phase, to withstand penetration into a layer obtained after drying the deposit formed after application of the first composition to a substrate.

The protocol is as follows:

Deposits are produced using the first composition, to a wet thickness of 100 μm, on the flat Bakelite sample holder of a contact angle tensiometer such as the DAT 1100 dynamic contact angle tensiometer sold by the company Fibro (Sweden).

To obtain such a deposit formed by applying the first composition, a rectangular metal stencil of dimensions 10×20 mm and 100 μm thick is laid flat on the sample holder of the tensiometer. The interior of the stencil frame is filled with the composition and the surface is leveled off using a standard microscope slide (glass slide, dimensions 26×76 mm), in order to remove the excess material.

After drying these deposits for 24 hours at room temperature (i.e. 23±2° C.) and a controlled relative humidity RH of 50±5%, the contact angle measurement are taken with the DAT 1100 machine at 20±1° C. and 50% relative humidity with water.

A drop of water, with a volume of between 2 and 10 microlitres, is deposited automatically by the machine onto the dry deposit formed by the first composition. The optical and image-analysis systems of the tensiometer make it possible to follow the change over time of the spreading of the drop. The contact angle of the liquid on the deposit formed by the first composition corresponds to the angle between the composition/liquid contact surface and the tangent to the drop passing through the point of encounter of the drop, the air and the surface. The software of the Fibro DAT 1100 tensiometer also makes it possible to monitor the change in volume of the drop.

Using the tensiometer coupled to the optical image-analysis system, the change in the contact angle θ and in the volume of the drop over one minute are monitored (it is generally possible to take a measurement every 20 ms).

There is considered to be no compatibility between the deposit formed by the first composition, having a low surface energy, and water, the main solvent of the second composition, when the following conditions are met:

V₃₀ₛ≧0.90V₀

θ_{0.30s}≧60°

with V₃₀ₛ being the volume of the drop of water 30 seconds after deposition and V₀ the volume of the drop instantaneously after depositing the drop (i.e. the first measurement acquisition at 20 ms and at most up to 0.100 second).

θ_{0.30s} is the contact angle measured after 30 seconds.

Second Composition

The second composition has a surface energy such that the difference between this surface energy and that of the first composition is greater than or equal to 5 mN/m.

According to one particular embodiment, this difference may be greater than or equal to 7 mN/m or even 10 mN/m.

The second composition also has a viscosity of between 0.001 and 2 Pa·s. Preferably, the viscosity is between 0.001 and 1 Pa·s. According to one particularly preferred embodiment, the viscosity is between 0.01 and 0.5 Pa·s.

According to one preferred embodiment, the second(composition is a composition with an aqueous continuous phase.

The expression “composition with an aqueous continuous phase” means that the composition has a conductivity, measured at 25° C., of greater than or equal to 23 μS/cm (microSiemens/cm), the conductivity being measured as indicated above.

According to one most particularly preferred embodiment, the second composition comprises a structuring agent and especially waxes as detailed hereinbelow.

Such a structuring agent may be present in the second composition in a content ranging for example from 1% to 20% by weight and especially front 0.5% to 10% by weight, for example from 1% to 10% by weight, relative to the total weight of the first composition.

Advantageously, the second composition also comprises a film-forming polymer, as detailed hereinbelow.

In all cases, when it is present, the content of film-forming polymer solid matters may range for example from 0.1% to 20% by weight especially from (0.5% to 15% by weight and preferably from 1% to 10% by weight relative to the total weight of the first composition.

According to one particular embodiment, the second composition is free of surfactant. It is also advantageously free of silicone waxes and/or fluoro waxes and also of pigments treated with a fluoro and/or silicone treatment.

Indeed, such compounds may be of a nature to counteract the surface energy desired for this second composition.

According to one preferred embodiment, the second composition comprises less than 10% and advantageously less than 5% by weight of wax relative to the total weight of the composition.

Thus, wax-free compositions are preferred, especially so as to avoid the use of surfactant.

According to another preferred embodiment, the second composition comprises fillers. The reason for this is that the presence of such fillers may constitute an aid for fixing the “excess material” as outlined previously.

Also, according to one particularly advantageous mode, the second composition contains low-density fillers.

For the purposes of the present invention, the term “low-density filler” means fillers whose tamped density, measured via the protocol given hereinbelow, range for example s from 0.01 to 0.80 g/cm³, especially ranging for example from 0.05 to 0.80 g/cm³ and in particular ranging for example from 0.10 to 0.80 g/cm³.

According to a particular embodiment, the surface energy of the second composition is greater than the one of the first composition.

Protocol for Measuring the Tamped Density (D_{T}):

A volume of between 240 and 250 ml of powder is poured into a 250 ml measuring cylinder having a mass M₀ (in g), using a funnel. The volume V₀ (in cm³) of powder poured in is then read on the cylinder and the cylinder filled with powder is weighed to measure the mass M₁ (in g).

The cylinder is then placed on the STAV 2003® machine from Stampf Volumeter. The cylinder is then subjected to a series of 2500 tamping blows, and the volume Vₙ (volume after the n^{th} series of 2500 tamping blows) of the powder in the cylinder is then measured at the end of each series.

Once Vₙ−Vₙ₊₁=<(2×Vₙ)/100, the tamping of the cylinder is stopped and the volume Vₙ (in cm³) is noted.

The tamped density is determined according to the following relationship:

D_{T}=(M₁−M₀)/Vₙ

Examples of tamped density values are given in the table below:

| | Tamped density |
|---|---|
| Low-density fillers | (g/cm³) |
| Sunsphere H-33 ® | 0.14 |
| from the company Asahi Glass | |
| Trefil Powder E-506C ® | 0.27 |
| from the company Dow Corning | |
| KSP-100 ® | 0.39 |
| from the company Shin-Etsu | |
| Silica Beads SB 700 ® | 0.48 |
| from the company Maprecos | |
| Sunsphere H-51 ® | 0.49 |
| from the company Asahi Glass | |
| Micropearl M-100 ® | 0.64 |
| from the company SEPPIC | |
| Plastic Powder D-400 ® | 0.76 |
| from the company Toshiki | |
| Polytrap 6603 Adsorber ® | 0.07 to 0.10 |
| from the company RP Scherrer | |

Among the low-density fillers, which are typically of spherical shape, mention may be made especially of:
silica microspheres, especially of open porosity, and in particular hollow silica microspheres, such as the products Silica Beads SP 700/HA® or Silica Beads SB 700® from the company Maprecos, Sunspheres H-33® and Sunspheres 11-51® from the company Asahi Glass, and Sunsil 130 from Sunjin Chemicals; these microspheres, where appropriate, may be impregnated with a cosmetic active agent.
microporous polymer microspheres, which have a structure similar to that of a sponge; they generally have a specific surface area of at least 0.5 m²/g and in particular of at least 1 m²/g, said specific surface area having no upper limit other than that resulting from the practical possibility of making microspheres of very high porosity: the specific surface area may be, for example, up to 1000 m²/g or even more. Illustrations of these microspheres that may be mentioned include acrylic polymer microspheres, such as those made of crosslinked acrylate copolymer Polytrap 6603 Adsorber® from the company RP Scherrer, and those made of polymethyl methacrylate Micropearl M 100® from the company SEPPIC,
polyurethane powder, such as the powdered copolymer of hexamethylene diisocyanate and of trimethylolhexyl lactone sold under the name Plastic Powder D-400® by the company Toshiki,
polymer microcapsules bearing only one closed cavity and form a reservoir, which may contain a liquid, especially a cosmetic active agent; they are prepared via known processes, such as those described in U.S. Pat. No. 3,615,972 and EP-A 0 56 219. They may be made, for example, of polymers or copolymers of ethylenically unsaturated acid, amine or ester monomers, of urea-formaldehyde polymers or of vinylidene chloride polymers or copolymers; by way of example, mention may be made of microcapsules made of methyl acrylate or methacrylate polymers or copolymers, or alternatively of copolymers of vinylidene chloride and of acrylonitrile, for instance Expancel® from the company Expancel; among these polymers, mention will be made especially of those containing 20-60% by weight of units derived from vinylidene chloride, 20-60% by weight of units derived from acrylonitrile and 0-40% by weight of other units such as units derived from an acrylic and/or styrene monomer; crosslinked acrylic polymers or copolymers may also be used,
elastomeric crosslinked organopolysiloxane powders, described especially in document JP-A-02 243 612, such as those sold under the name Trefil Powder E-506C by the company Dow Corning, elastomeric crosslinked organopolysiloxane powders coated with silicone resin, especially silsesquioxane resin, as described, for example, in U.S. Pat. No. 5,538,793. Such elastomers are sold under the names KSP-100, KSP-101, KSP-102, KSP-103, KSP-104 and KSP-105 by the company Shin-Etsu, and
mixtures thereof.

Mention may also be made of concave particles in the form of hollow sphere portions constituted of an organosilicone material, for instance the particles constituted by the bowl-shaped crosslinked organosilicone TAK-110 (methylsilanol/silicate crosslinked polymer) sold by the company Takemoto Oil & Fat.

The second composition according to the invention may comprise a low-density filler or mixture of fillers in a content ranging for example from 0.1% to 50%, by weight, especially from 0.5% to 40% by weight and in particular from 1% to 30% by weight relative to the total weight of the second composition.

Besides the abovementioned ingredients, the second composition included in the makeup kit according to the invention may comprise other additives that are standard in the cosmetics field, with the proviso that their respective amounts do not harm the characterization by the surface energy and the viscosity of the second composition.

Thus, when it is present in the form of a composition with an aqueous continuous phase, the second composition may comprise a water-soluble gelling agent as described later.

The water-soluble film-forming polymers mentioned below may also act as water-soluble gelling agent. In this respect, mention may be made, for example, of hydroxyethylcellulose and gum arabic.

The water-soluble gelling polymer may be present in the second composition in a solid matter content ranging for example from 0.01% to 60% by weight, preferably from 0.25% to 40% by weight, better still from 0.5% to 30% by weight or even from 1% to 20% by weight relative to the total weight of the second composition.

According to another embodiment, the second composition is anhydrous.

Oils

The first and second compositions included in the makeup kit according to the invention may comprise one or more oils or organic solvent.

The oil(s) present in the composition of the invention may be chosen from volatile oils and/or non-volatile oils, and mixtures thereof.

The expression “volatile oil or organic solvent” means an oil or organic solvent (or non-aqueous medium) capable of evaporating on contact with the skin in less than one hour, at room temperature and atmospheric pressure.

The volatile oil is a volatile cosmetic oil, which is liquid at room temperature, especially having a non-zero vapour pressure, at room temperature and atmospheric pressure, in particular having a vapour pressure ranging for example from 0.13 Pa to 40 000 Pa (10⁻³ to 300 mmHg) and preferably ranging for example from 1.3 Pa to 8000 Pa (0.01 to 60 mmHg).

In particular, the volatile oils are chosen from oils with all evaporation rate greater than or equal to 0.002 mg/cm²/min. The evaporation rate is measured as follows:

15 g of oil or of the mixture of oils to be tested are introduced into a crystallizing dish (diameter: 7 cm) placed on a scale located in a chamber of about 0.3 m³ with a regulated temperature (25° C.) and hygrometry (50% relative humidity).

The liquid is allowed to evaporate freely, without stirring, ventilation being provided by means of a ventilator (operating at 2700 rpm and of dimensions 80×80×42 mm, for example the reference 8550 N from Papst-Motoren, the flow rate corresponds to about 50 m³/hour) arrange for example d vertically above the crystallizing dish containing the solvent, the vanes being directed towards the crystallizing dish and 20 cm away from the base of the crystallizing dish.

The mass of oil(s) remaining in the crystallizing dish is measured at regular intervals. The evaporation rates are expressed as mg of oil evaporated per unit of surface area (cm²) and per unit of time (minutes).

The volatile oils (or organic solvents) may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar® or Permethyl®, branched C₈-C₁₆ esters and isohexyl neopentanoate, and mixtures thereof. Other volatile hydrocarbon-based oils, for instance petroleum distillates, especially those sold under the name Shell Solt® by the company Shell, may also be used.

Volatile oils that may also be used include volatile silicones, for instance volatile linear or cyclic silicone oils, especially those with a viscosity ≦6 centistokes (6×10⁻⁶ m²/s) and especially containing from 3 to 6 silicon atoms, these silicones optionally comprising one or more alkyl or alkoxy groups containing from 1 or 2 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, 3-butyl-1,1,1,3,5,5,5-heptamethyltrisiloxane. 3-propyl-1,1,1,3,5,5,5-heptamethyltrisiloxane and 3-ethyl-1,1,1,3,5,5,5-heptamethyltrisiloxane, and mixtures thereof.

Volatile organic solvents, especially fluorinated solvents such as nonafluoromethoxybutane or perfluoromethylcyclopentane, may also be used.

The non-volatile silicone oils that may be used in the first and second compositions according to the invention may be non-volatile polydimethylsiloxanes (PDMSs), polydimethylsiloxanes comprising alkyl or alkoxy groups that are pendent and/or at the end of a silicone chain, the groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

The fluoro oils that may be used in the first and second compositions according to the invention are, in particular, fluorosilicone oils, fluoro polyethers or fluorosilicones, as described in document EP-A-847 752.

Advantageously, the first composition comprises at least one volatile oil.

Advantageously, the volatile oil(s) is (are) chosen from volatile hydrocarbon-based oils containing from 8 to 16 carbon atoms, such as isododecane, volatile silicone oils such as decamethylcyclopentasiloxane (D5) and dodecamethylcyclohexasiloxane (D6), and mixtures thereof.

The first and second compositions according to the invention may also comprise at least one non-volatile compound, which is water-insoluble and liquid at room temperature, especially at least one non-volatile organic solvent or oil, which may be chosen in particular from non-volatile hydrocarbon-based oils and/or silicone oils and/or fluoro oils.

Non-volatile hydrocarbon-based oils that may especially be mentioned include:
hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stéarineries Dubois or those sold under the names Miglyol 810®, 812® and 818® by the company Dynamit Nobel,
synthetic ethers containing from 10 to 40 carbon atoms.
linear or branched hydrocarbons of mineral or synthetic origin, such as petroleum jelly, polydecenes, hydrogenated polyisobutene such as parleam, and squalane, and mixtures thereof,
synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (ectostearyl octanoate), isopropyl myristate, isopropyl palmitate, C₁₂ to C₁₅ alcohol benzoate, hexyl laurate, disopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters,
fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
higher fatty acids such as oleic acid, linoleic acid or linolenic acid,

and mixtures thereof.

The non-volatile silicone oils that may be used in the first and second compositions according to the invention may be non-volatile polydimethylsiloxanes (PDMS), polydimethylsiloxanes comprising alkyl or alkoxy groups, which are pendent and/or at the end of a silicone chain, these groups each contain from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyltrimethylsiloxysilicates.

The fluoro oils that may be used in the first and second compositions of the invention are especially fluorosilicone oils, fluoro polyethers or fluoro silicones as described in document EP-A-847 752.

Structuring Agent

The first and second compositions included in the makeup kit according to the invention may comprise at least one agent for structuring the oily phase or organic solvent, chosen from waxes, semi-crystalline polymers and lipophilic gelling agents, and mixtures thereof.

The amount of oily structuring agent may be adjusted by a person skilled in the art as a function of the structuring properties of said agents.

Wax(es)

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than or equal to 45° C. and in particular greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging for example from −20° C. to 100° C., at a heating rate of 10° C./minute, it is then cooled from 100° C. to −20° C. at a cooling rate of 10° C./minute and is finally subjected to a second temperature increase ranging for example from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that are suitable for use in the invention are chosen from waxes that are solid at room temperature of animal, plant, mineral or synthetic origin, and mixtures thereof.

The waxes that may be used in the cosmetic compositions generally halve a hardness ranging for example from 0.01 MPa to 15 MPa, especially greater than 0.05 MPa and in particular greater than 0.1 MPa.

The hardness is determined by measuring the compression force, which is measured at 20° C. using the texturometer sold under the name TA-XT2 by the company Rheo, equipped with a stainless-steel cylinder 2 mm in diameter, travelling at a measuring speed of 0.1 mm/second, and penetrating into the wax to a penetration depth of 0.3 mm.

The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

As illustrations of waxes that are suitable for the invention mention may be made especially of hydrocarbon-based waxes, for instance beeswax, lanolin wax, Chinese insect waxes, rice bran wax, carnauba wax, candelilla wax, ouricurry wax, esparto grass wax, berry wax, shellac wax, Japan wax and sumach wax; monitan wax, orange for example wax and lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylolpropane) tetrastearate sold under the name Hest 2T-4S® by the company Heterene.

The waxes obtained by transesterification and hydrogenation of plant oils, such as castor oil or olive oil, for instance the waxes sold under the names Phytowax ricin 16L64® and 22L73® and Phytowax Olive 18L57 by the company Sophim, may also be used. Such waxes are described in patent application FR-A-2 792 190.

In the present invention, waxes provided in the form of small particles having a size expressed as the mean “effective” volume diameter D[4.3] of about from 0.1 to 20 micrometres, in particular from 0.2 to 10 micrometres and more particularly from 0.5 to 1 micrometre, which are referred to hereinafter as “micro waxes”, may also be used. For the purpose of distinction, the waxes used according to the invention in the form of fragments of larger size are referred to hereinbelow as “conventional waxes”.

The particle sizes may be measured by various techniques: mention may be made in particular of light-scattering techniques (dynamic and static). Coulter counter methods, sedimentation rate measurements (related to the size via Stokes' law) and microscopy. These techniques make it possible to measure a particle diameter and, for some of them, a particle size distribution.

The sizes and size distributions of the particles in the cosmetic compositions are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The cosmetic composition is characterized by its mean “effective” volume diameter D[4.3], defined in the following manner:

$$D [ 4.3 ] = \frac{{\sum }_{i} {V}_{i} \cdot {d}_{i}}{{\sum }_{i} {V}_{i}}$$

in which V₁ represents the volume of the particles with an effective diameter d₁. This parameter is described especially in the technical documentation of the granulometer.

The measurements are performed at 25° C. on a dilute particle dispersion, obtained from the composition in the following manner: 1) dilution by a factor of 100 (with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

As microwaxes that may be used in the first and second compositions according to the invention, mention may be made of carnauba microwaxes, such as the product sold under the name MicroCare 350® by the company Micro Powders, synthetic microwaxes, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and(of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene micropowders such as the products sold under the names Microslip 519® and 519D® by the company Micro Powders.

In the first and second compositions included in the makeup kit according to the invention, it is obviously possible to use a mixture of waxes and especially to use one or more waxes of conventional type and one or more waxes known as micro waxes.

Lipophilic Gelling Agents

The gelling agents that may be used in the first and second compositions included in the makeup kits according to the invention may be organic or mineral, polymeric or molecular lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

Mention may also be made of fumed silica optionally subjected to a hydrophobic surface treatment, the particle size of which is less than 1 μm. Indeed, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:
trimethylsiloxyl groups, which are obtained especially by treating fumed silica in the presence of hexamethyldisilazane. Silicas thus treated are known as Silica silylate according to the CTFA (6th edition, 1995). They are sold, for example, under the references Aerosil R812® by the company Degussa, and (Cab-O-Sil TS-530® by the company Cabot;
dimethylsilyloxyl or polydimethylsiloxane groups, which are obtained especially by treating fumed silica in the presence of polydimethylsiloxane or dimethyldichlorosilane. Silicas thus treated are known as silica dimethyl silylate according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The hydrophobic fumed silica preferably has a particle size that may be nanometric to micrometric, for example ranging for example from about 5 to 200 nm.

The polymeric organic lipophilic gelling agents are, for example, partially or totally crosslinked elastomeric organopolysiloxanes of three-dimensional structure for instance those sold under the names KSG6®, KSG16® and KSG18® from Shin-Etsu. Trefil E-505C® or Trefil E-506C® from Dow Corning, Gransil SR-CYC®, SR DMF 10®, SR-DC556®, SR 5CYC Gel®, SR DMF 10 Gel® and SR DC 556 Gel® from Grant Industries and SF 1204® and JK 113® from General Electric; ethylcellulose, for instance the product sold under the name Ethocel by Dow Chemical; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name Uniclear 100 VG® by the company Arizona Chemical; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof. Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylenepropylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name VersaGel®, for instance the mixture of butylene/ethylene/styrene triblock copolymer and of ethylene/propylene/styrene star copolymer in isododecane (Versagel M 5960).

Among the gelling agents that may be used in the cosmetic compositions included in the makeup kits according to the invention, mention may also be made of fatty acid esters of dextrin, such as dextrin palmitates, especially the products sold under the name Rheopearl TL® or Rheopearl KL® by the company Chiba Flour.

Film-Forming Polymer

According to one particular embodiment, the first and second compositions included in the makeup kit according to the invention may comprise at least one film-forming polymer.

In the present invention, the expression “film-forming polymer” means a polymer that is capable, by itself or in the presence of an auxiliary film-forming agent, of forming a macroscopically continuous film that adheres to the keratin fibres, preferably a cohesive film and better still a film whose cohesion and mechanical properties are such that the film can be isolated and manipulated separately, for example when the film is made by casting on a non-stick surface, for instance a Teflon-coated or silicone-coated surface.

Among the film-forming polymers that may be used in their first and second compositions included in the makeup kit according to the invention, mention may be made of synthetic polymers, of radical type or of polycondensate type, and polymers of natural origin, and mixtures thereof.

Liposoluble Polymers

According to one embodiment variant, the film-forming polymer may be a polymer dissolved in the oily phase comprising oils or organic solvents such as those mentioned previously (the film-forming polymer is then the to be a liposoluble polymer).

Examples of liposoluble polymers that may be mentioned include copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether in which the alkyl group comprises from 2 to 18 carbon atoms) or an allylic or methallylic ester (containing a saturated, linear or branched hydrocarbon-based radical of 1 to 19 carbon atoms linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate, 1-octadecene, vinyl acetate/1-dodecene, vinyl stearate/ethyl vinyl ether, vinyl propionate cetyl vinyl ether, vinyl stearate/allyl acetate, vinyl 2,2-dimethyloctanoate vinyl laurate allyl 2,2-dimethylpentanoate/vinyl laurate, vinyl dimethylpropionate/vinyl, stearate, allyl dimethylpropionate/vinyl stearate, vinyl propionate/vinyl stearate, crosslinked with 0.2% divinylbenzene, vinyl dimethylpropionate/vinyl laurate, crosslinked (with 0.2%) divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene and allyl propionate/allyl stearate, crosslinked with 0.2% divinylbenzene.

Examples of liposoluble film-forming polymers that may also be mentioned include liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from copolymers of polyvinyl stearate, polyvinyl stearate crosslinked with the aid of divinylbenzene, of diallyl ether or of diallyl phthalate, copolymers of polystearyl(meth)acrylate, polyvinyl laurate and polylauryl(meth)acrylate, it being possible for these poly(meth)acrylates to be crosslinked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging for example from 2 000 to 500 000 and preferably from 4 000 to 200 000.

As liposoluble film-forming polymers that may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsequioxane resins that may be mentioned include those sold:
by the company Wacker under the reference Resin MK, such as Belsil PMS MK;
by the company Shin-Etsu under the reference KR-220L.

Siloxysilicate resins that may be mentioned include trimethyl siloxysilicate (TMS) resins such as those sold under the reference SR 1000 by the company (General Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Silicone polyamides of the polyorganosiloxane type may also be used, such as those described in documents U.S. Pat. No. 5,874,069, U.S. Pat. No. 5,919,441, U.S. Pat. No. 6,051,216 and U.S. Pat. No. 5,981,680.

These silicone polymers may, for example, belong to the following two families:

1) polyorganosiloxanes comprising at least two groups capable of establishing hydrogen interactions, these two groups being located in the polymer chain, and/or

2) polyorganosiloxanes comprising at least two groups capable of establishing hydrogen interactions, these two groups being located on grafts or branches.

According to one embodiment of the invention, the film-forming polymer is a film-forming linear block ethylenic polymer, which preferably comprises it least a first block and at least a second block with different glass transition temperatures (Tg), said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in document EP 1 411 069 or WO 04/028 488.

Water-Soluble Polymers

According to another embodiment variant, the film-forming polymer may be a water-soluble polymer. The polymer is then solubilized in the aqueous phase of the composition. Examples of water-soluble film-forming polymers that may be mentioned include:
proteins, for instance proteins of plant origin such as wheat proteins and soybean proteins; proteins of animal origin such as keratins, for example keratin hydrolysates and sulfonic keratins;
polymers of cellulose such as hydroxyethylcellulose, hydroxypropylcellulose, methylcellulose and ethylhydroxyethylcellulose;
acrylic polymers or copolymers, such as polyacrylates or polymethacrylates;
vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;
polymers of natural origin, which are optionally modified, such as:
gum arabics, guar gum, xanthan derivatives, karaya gum,
alginates and carrageenans;
glycosaminoglycans, hyaluronic acid and derivatives thereof shellac resin, sandarac gum, dammar resins, elemi gum and copal resins;
desoxyribonucleic acid;
mucopolysaccharides such as chondroitin sulfate,

and mixtures thereof.

Polymers of Natural Origin

The polymers of natural origin, optionally modified, may be chosen from shellac resin, sandarac gum, dammar resin, elemi gum, copal resin and cellulose-based polymers, and mixtures thereof.

Polymers in Dispersed Form

The film-forming polymer may be present in the form of particles dispersed in an aqueous phase or in a non-aqueous solvent phase, which is generally known as a latex or pseudolatex. The techniques for preparing these dispersions are well known to those skilled in the art.

a) Aqueous Dispersion

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo, Syntran 5760® by the company Interpolymer, Allianz Opt® by the company Röhm & Haas, or the aqueous dispersions of polyurethane sold under the names Neorez R-981® and Neorez R-974® by the company Avecia-Neoresins, Avalure UR-405®, Avalure-UR-410®, Avalure UR-425®, Avalure UR-450®, Sancure 875®, Avalure UR-445® and Sancure 2060® by the company Noveon, Impranil 85® by the company Bayer and Aquamere 11-1511® by the company Hydromer; the sulfopolyesters sold under the brand name “Eastman AQ®” by the company Eastman Chemical Products, vinyl dispersions, for instance Mexomer PAM®, aqueous dispersions of polyvinyl acetate, for instance Vinybran® from the company Nisshin Chemical or those sold by the company Union Carbide, aqueous dispersions of vinylpyrrolidone terpolymer, dimethylaminopropymethacrylamide and lauryldimethylpropylmethacrylamidoammonium chloride terpolymer such as Styleze W from ISP, aqueous dispersions of polyurethane/polyacrylic hybrid polymers such as those sold under the references Hybridur® by the company Air Products or Duromer® from National Starch, dispersions of core/shell type: for example those sold by the company Atofina under the reference Kynar (core: fluoro, shell: acrylic) or alternatively those described in document U.S. Pat. No. 5,188,899 (core: silica, shell: silicone), and mixtures thereof.

b) Non-Aqueous Dispersion

Mention may also be made of dispersions of particles of a grafted ethylenic polymer, preferably an acrylic polymer, in a liquid oily phase, the ethylenic polymer advantageously being dispersed in the absence of additional stabilizer at the surface of the particles as described especially in document WO 04/055 081.

The first and second compositions in accordance with the invention may comprise a plasticizer that promotes the formation of a film with the film-forming polymer. Such a plasticizer may be chosen from any compound known to those skilled in the art as being capable of fulfilling the desired function.

In this respect, mention may be made of acrylic dispersions in isododecane, for instance Mexomer PAP® from the company Chimex.

Water and/or Water-Soluble Solvent

In the present invention, the term “water-soluble solvent” denotes a compound that is liquid at room temperature and water-miscible (miscibility in water of greater than 50% by weight at 25° C. and atmospheric pressure).

Among the water-soluble solvents that may be used in said cosmetic compositions, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and/or water-soluble solvent(s)) may be introduced, as such, into the first and second compositions included in the makeup kit or may be incorporated therein by means of one or more ingredients constituting said first and second compositions. Thus, water may especially be introduced into one of the composition by means of introducing a latex or a pseudolatex, i.e. an aqueous dispersion of polymer particles.

Dyestuffs

The first and second compositions included in the makeup kits according to the invention may also comprise, independently of each other, at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D & C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue Or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange for example 5, quinoline yellow and annatto.

These dyestuffs may be present in a content ranging for example from 0.01% to 30% by weight relative to the total weight of each of said first and second compositions.

The dyestuffs may be identical or different in the first and second compositions. It is also possible, by varying the shades of the compositions to personalize the makeup results at will.

Thus, according to one embodiment, the compositions may have different shades. For example, one of the compositions may be transparent or translucent, and the other composition coloured. According to one variant, the first and second compositions may have different colours.

According to one variant, the second composition may advantageously comprise nacres or flakes, such as reflective particles.

The two compositions may also differ by any optical characteristic visible to the naked eye, such as gloss.

Additives

Fibres

The first and second compositions included in the makeup kits according to the invention may, independently of each other, also comprise fibres to especially allow an improvement in the lengthening effect in the field of making up keratin fibres.

The term “fibre” should be understood as meaning an object of length L and diameter D such that L is very much greater than D, D being the diameter of the circle in which the cross section of the fibre is inscribed. In particular, the ratio L/D or shape factor) is chosen in the range for example from 3.5 to 2500, especially from 5 to 500 and in particular from 5 to 150.

The fibres that may be used in the compositions included in the make up kits of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular their ends are blunt and/or polished to prevent injury.

In particular, the fibres have a length ranging for example from 1 μm to 10 mm, preferably from 0.1 mm to 5 mm and better still from 0.3 mm to 3.5 mm. Their cross section may be within a circle of diameter ranging for example from 2 nm to 500 μm, preferably ranging for example from 100 nm to 100 μm and better still from 1 μm to 50 μm. The weight or yarn count of the fibres is often given in denier or decitex, and represents the weight in grams per 9 km of yarn. In particular, the fibres may have a yarn count chosen in the range for example from 0.15 to 30 denier and better still from 0.18 to 18 denier.

The fibres that may be used in the compositions included in the makeup kits according to the invention may be chosen from rigid and non-rigid fibres, and may be of synthetic or natural, mineral or organic origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramide) fibres sold especially under the name Kevlar® by the company DuPont de Nemours.

The fibres may be present in the first or second composition in a content ranging for example from 0.01% to 10% by weight, in particular from 0.05% to 5% by weight and more particularly from 0.1% to 3% by weight relative to the total weight of the composition.

In reality, when they are present, the fibres are preferentially present in the first composition.

Cosmetic Active Agents

The first and second compositions included in the makeup kit according to the invention may also, independently of each other, comprise any ingredient conventionally used in cosmetics. These ingredients may be chosen especially from polymers, especially fixing polymers; opacifiers; fragrances; thickeners; gelling agents; hair dyes; silicone resins; silicone gums; preserving agents; antioxidants; cosmetic active agents, sunscreens; pH stabilizers; vitamins; moisturizers, and mixtures thereof. The amounts or these various ingredients are those conventionally used in the fields under consideration, for example from 0.01% to 20% by weight relative to the total weight of the first composition, and from 0.1% to 10% by weight relative to the total weight of the second composition.

Needless to say, a person skilled in the art will take care to select this or these optional additive(s), and/or the amount thereof, such that the advantageous properties of the first and second compositions according to the invention are not, or are not substantially, adversely affected by the envisaged addition.

Applicator and Kit

Any type of applicator may be used for the purpose of depositing the first and/or the second composition.

The respective applicators of the first and second compositions may be identical or different.

According to one variant of the invention, the first and second compositions may be applied by means of two different applicating portions of the same applicator.

According to another variant, the makeup kit according to the present invention comprises one or more means for applying the first and second compositions.

The first and second cosmetic compositions included in the makeup kit of the invention may in particular be applied to the eyelashes using a brush, a comb, a threaded stem or a pen equipped with a toothed thumbwheel.

In the present case, the first composition may be applied using any applicator, provided that it allows localized deposition on part of the length of the keratin fibre, and, when applied to an eyelash, on ⅔ of its length at most.

It is particularly advantageous to perform the application of the first composition with a comb.

It is particularly advantageous to perform the application of the second composition with a threaded stem or a pen equipped with a toothed thumbwheel.

The application of the second composition using a threaded stem especially makes it possible to form fine droplets more or less uniformly aligned along the end of the eyelashes when the first composition has been applied to the base of the eyelashes.

The threaded stem may be made of plastic material or of metal or of any other suitable material.

The pen equipped with a toothed thumbwheel is more particularly described in U.S. Pat. No. 5,851,019 or FR 2 588 733.

This pen allows an accurate dosage of products to be applied at the outlet of the pen after actuating a toothed thumbwheel or a push-button that advances a piston on each rotation/press.

This application means especially allows the formation of drops, for example of single drops at the end of the eyelashes when the first composition has been applied to the base of the eyelashes. The drops are generally of larger size than those obtained by application using the threaded stem.

The makeup kit according to the invention may, according to one particular embodiment, comprise at least two separate packagings, one comprising the first composition and the other comprising the second composition.

In reality, since the keratin fibres are made up by means of multiple actions by the user, i.e. in at least two steps, the first consisting in applying the “base coat” composition and the second consisting in applying the “top coat” composition totally or partially over the first “base coat” composition and onto the keratin fibres, a makeup kit conditioned in a single packaging is particularly suitable for use. This alternative constitutes a preferred embodiment of the invention. According to one particularly advantageous embodiment, this packaging article is equipped with two separate applicators.

When the kit is in the form of a single packaging it may be presented as a container delimiting at least one compartment or reservoir that comprises the “base coat” composition, said compartment being closed by means of a closing member, and at least one compartment or reservoir that comprises the “top coat” composition, also being closed by means of a closing member.

The applicator may be integrally attached to the container. Advantageously the applicator is integrally attached to a stem which, itself, is integrally attached to the closing member.

The closing member may be coupled to the container by screwing. Alternatively, the coupling between the closing member and the container takes place other than by screwing, especially via a bayonet mechanism, by click-fastening or by tightening. The term “click-fastening” in particular means any system involving the passing of a rim or bead of material by elastic deformation of a portion, especially of the closing member, followed by return to the elastically unstressed position of said portion after the rim or bead has been passed.

The container, advantageously comprising two compartments or reservoirs, may be at least partly made of thermoplastic material. Examples of thermoplastic materials that may be mentioned include polypropylene and polyethylene.

Alternatively, the container is made of a non-thermoplastic material, especially of glass or metal (or alloy).

The container is preferably equipped with a drainer located in the region of at least one aperture of the container. Such a drainer makes it possible to wipe the applicator and, optionally, the stem to which it may be solidly attached. Such a drainer is described, for example, in patent FR 2 792 618.

According to one particularly preferred embodiment, the makeup kit comprises two reservoirs each comprising one of the “base coat” and “top coat” compositions, each of the reservoirs being equipped with a brush and/or a comb and/or a threaded stem and/or a pen equipped with a toothed thumbwheel, in particular the reservoir of the “base coat” composition being equipped with a mascara comb and the reservoir of the “top coat” composition being equipped with a threaded stem or a pen equipped with a toothed thumbwheel.

Makeup and/or Care Process

The first composition may be applied to all or part of the keratin fibres, for example to half or a third of the length of the fibres, and in all cases, when applied to eyelashes, to two thirds of their length at most.

Thus, when it is applied to the base of the eyelashes, the makeup result is similar to that represented in FIGS. 2 and 3.

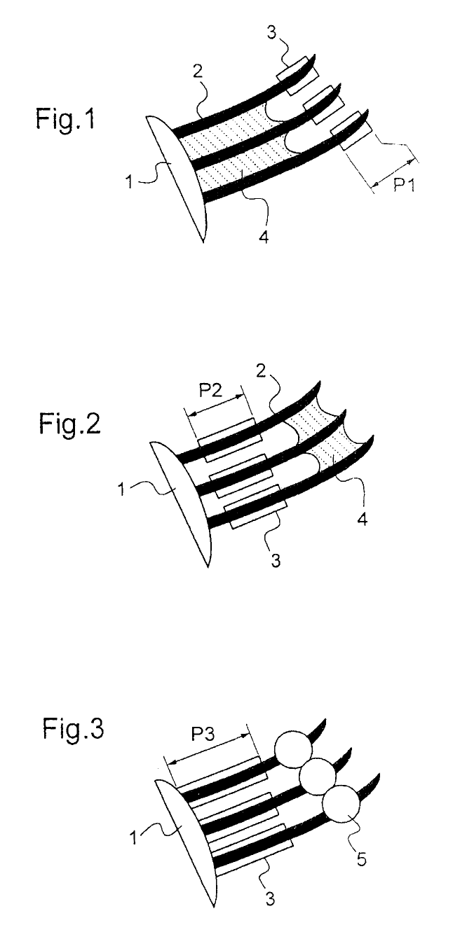

When it is applied to the end of the eyelashes, the makeup result is similar to that represented in FIG. 1.

The second composition may also be applied to all or part of the keratin fibres, for example to half or a third of the length of the fibres. It may be applied before the deposit formed by the first composition has totally dried.

For example, the second composition may be deposited onto the entire keratin fibre, and it is found, surprisingly, that the deposition of this second composition dewets from the surface onto which the first composition has been deposited, in order preferentially to make up the areas of keratin fibres not covered by the first deposit.

The present invention thus also relates to a process for making up and/or for non-therapeutically caring for keratin fibres, wherein it comprises at least one step of applying a first composition in accordance with the present invention to all or part of the keratin fibres, forming a first deposit, and at least one step of applying a second composition in accordance with the present invention onto all or part of the first deposit and/or of the keratin fibres.

The present invention is also directed towards a process for making up and/or for non-therapeutically caring for keratin fibres, which is intended to form pearls or bridges at the end of said keratin fibres on said keratin fibres, wherein it comprises it least one step of applying to the base of the keratin fibres a first composition in accordance with the present invention forming a first deposit, and at least one step of applying a second composition in accordance with the present invention onto all or part of the first deposit and/or of the keratin fibres.

Finally, the present invention is directed towards a process for making up and/or for non-therapeutically caring for keratin fibres, which is intended to form bridges close to the base of said keratin fibres, wherein it comprises at least one step of applying to the end of the keratin fibres a first composition in accordance with the present invention forming a first deposit and at least one step of applying a second composition onto all or part of the first deposit and/or of the keratin fibres.

The invention may be understood more clearly on examining the attached drawing, in which:

FIG. 1 represents a portion of eyelash fringes made up according to one of the alternatives of the makeup process in accordance with the present invention,

FIG. 2 represents a portion of eyelash fringes made up according to another alternative of the makeup process in accordance with the present invention

FIG. 3 represents a portion of eyelash fringes made up according to yet another alternative of the makeup process in accordance with the present invention.

In all the figures, a portion of eyelid 1 is represented.

In FIG. 1, the makeup has been obtained by applying the first composition to the end of the eyelashes over a portion P1 which, in FIG. 1, corresponds to about a third of the length of said eyelash. This length may be brought to two thirds of the length of said eyelash without changing the type of makeup. The second composition has been applied to the entire length of the eyelashes. A bridge of material 4 has been created between the eyelashes at their base.

In FIGS. 2 and 3, the makeup has been obtained applying the first composition to a portion P2 or P3, respectively, of the eyelashes, which in FIG. 2 or 3, respectively, corresponds to about the inner third of the length of said eyelash.

This portion P2 or P3, respectively, may be brought to two thirds of the length of said eyelash without changing the type of makeup. The second composition has been applied to the entire length of the eyelashes. A bridge of material 4 or pearls 5, respectively, has (have) been created between the eyelashes at their end.

In the case of making up the eyelashes, such a process may be performed on all or part of an eyelash fringe.

The formulation examples that follow are given as non-limiting illustrations of the invention. Unless otherwise mentioned, the amounts are given as mass percentages.

### EXAMPLES

The following compositions may be prepared:

Composition 1: waterproof mascara formulation containing an alkyl silicone and fluoro-coated pigments:
17% C3045 alkyl dimethicone SF-1642 (GE-Bayer Silicones)
7% allyl stearate/vinyl acetate copolymer
5% black iron oxide coated with perfluoroalkyl phosphate sold under the name Black Iron Oxide Covafluor by the company Wackherr
5% Quaternium-18 Bentonite (Bentone 38V® from Elementis)
1.6% propylene carbonate
64.4% isododecane

Composition 2: mascara formulation containing a hollow hydrophilic silica:
1.4% hydroxyethylcellulose (Cellosize QP 4400H from Union Carbide)
25% hollow hydrophilic silica (Sunsil 130 from Sunjin Chemicals)
6.69% gum arabic
3.35% pigments (black iron oxide)
3.35% propylene glycol
0.30% sodium methyl paraben (preserving agent)
water qs 100 g

The surface energy of composition 1 was measured according to the protocol described above for the “first composition”. The measured value is 13.73 mN/m.

The surface energy of composition 2 was measured according to the protocol described above for the “second composition”. The measured value, over a mean of three measurements, is 52.83 mN/m.

Composition 3: waterproof mascara formulation containing a Teflon-coated wax and PDMS-coated pigments:
20% micronized polytetrafluoroethylene powder sold under the name Microslip 519L by the company MicroPowders
7% allyl stearate/vinyl acetate copolymer
5% brown iron oxide coated with silica and with polydimethylsiloxane, sold under the name SA-Luce Red by the company Lyoshi Kasei
8% Quaternium-18 Bentonite (Bentone 38VO from Elementis)
2.6% propylene carbonate
57.4% isododecane

Composition 4: mascara formulation containing hollow PMMA microspheres and a latex:
1.4% hydroxyethylcellulose (Cellosize QP 440011 from Union Carbide)
15% hollow polymethyl methacrylate microspheres (Covabead LH 85 from LCW)
6.69% gum arabic
10% ethyl acrylate/methyl methacrylate crosslinked copolymer, as an aqueous 50% dispersion, sold under the name Daitosol 5000 AD by the company Daito Kasei Kogyo
3.35% pigments (brown iron oxide)
3.35% propylene glycol
0.30% sodium methyl paraben (preserving agent)
water qs 100 g

Various makeups are tested on examples of false eyelashes using, in pairs, compositions 1 to 4 above.

When composition 1 or 3 is applied to the base of the eyelashes and composition 2 or 4 to the entire eyelash fringe, there is dewetting of composition 2 or 4, which migrates towards the end of the eyelashes (area of higher surface energy), so as to approach the makeup result of FIG. 3.

Moreover, composition 1 may be applied as a base coat and composition 4 as a top coat.

When composition 1 or 3 is applied to the end of the eyelashes, there is dewetting of composition 2 or 4, which migrates towards the areas not made up with composition 1 or 3 (middle and base of the eyelashes) so as to approach the makeup result of FIG. 1.

Composition 1 may be applied as a base coat and composition 2 as a top coat.

Alternatively, composition 3 may be applied as a base coat and composition 2 as a top coat.

Also alternatively, composition 3 may be applied as a base coat and composition 4 as a top coat.

The above written description of the invention provides a manner and process of making and using it such that any person skilled in this art is enabled to make and use the same, this enablement being provided in particular for the subject matter of the appended claims, which make up a part of the original description and including a keratin fibre makeup kit comprising at least:

a first composition with a surface energy of less than or equal to 30 mN/m at 23° C.±2° C., and

a second composition with a surface energy such that the difference between this surface energy and that of the first composition is greater than or equal to 5 mN/m, and a viscosity of between 0.001 and 2 Pa·s at 25° C.±0.5° C.

With regard to the surface energy of the second composition, a preferred embodiment includes the second composition having a surface energy such that the difference between the surface energy of the second composition and that of the first composition is greater than or equal to 5 mN/m.

Further with regard to the surface energy of the second composition, a preferred embodiment includes the second composition having a surface energy that is at least 5 mN/m greater than the surface energy of first composition.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. pre-face examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not to be limited to the embodiments shown but is to be accorded the widest scope consistent with the principles and features disclosed herein. In this regard, certain embodiments within the invention may not show every benefit of the invention, considered broadly. Thus, although the present invention has been described with reference to particular embodiments, it is to be understood that these embodiments are merely illustrative of the principles and applications of the present invention. It is therefore to be understood that numerous modifications may be made to the illustrative embodiments and that other arrangements for example may be devised without departing from the spirit and scope of the present invention as defined by the appended claims.

1. A keratin fibre makeup kit comprising at least:
a first composition having a surface energy of less than 1 or equal to 30 mN/m at 23° C.±2° C., and
a second composition having a surface energy such that the difference between the surface energy of the second composition and that of the first composition is greater than or equal to 5 mN/m, and a viscosity of between 0.001 and 2 Pa·s at 25° C.±0.5° C.

2. The kit according to claim 1, wherein the first composition has a surface energy of less than or equal to 25 mN/m.

3. The kit according to claim 1, wherein the first composition comprises silicone and/or fluoro compounds selected from the group consisting of fluoro waxes and/or silicone waxes, pigments with a silicone and/or fluoro surface coating or treatment, and mixtures thereof.

4. The kit according to claim 3, wherein the first composition comprises fluoro waxes and/or silicone waxes selected from the group consisting of fluoro esters, microdispersions of fluoro waxes, substituted linear polysiloxanes, alkoxy dimethicones, (C₂₀-C₆₀) alkyl dimethicones, and hydrocarbon-based waxes modified with silicone or fluoro groups.

5. The kit according to claim 1, wherein the first composition comprises from 0.1% to 50% by weight of fluoro waxes and/or silicone waxes relative to the total weight of the composition.

6. The kit according to claim 1, wherein the first composition comprises pigments treated with a fluoro and/or silicone treatment.

7. The kit according to claim 6, wherein the pigments treated with a fluoro and/or silicone treatment are selected from the group consisting of pigments coated with a perfluoroalkyl phosphate, a polyhexafluoropropylene oxide, and pigments coated with a silicone compound, for instance methicones, dimethicones, polyorganosiloxanes comprising perfluoroalkyl, perfluoropolyether and perfluoroalkyl silane groups.

8. The kit according to claim 1, wherein the first composition Comprises from 0.01% to 30% by weight, of pigments treated with a fluoro and/or silicone treatment relative to the total weight of the composition.

9. The kit according to claim 1, wherein the first composition comprises an oily continuous phase.

10. The kit according to claim 1, wherein the first composition comprises a content of volatile oil greater than its content of non-volatile oil.

11. The kit according to claim 1, wherein the total oil content in the first composition is 30% to 90% by weight, relative to the total weight of the first composition.

12. The kit according to claim 1, wherein the first composition is anhydrous.

13. The kit according to claim 1, wherein the first composition comprises at least one structuring agent selected from the group consisting of waxes other than fluoro waxes and silicone waxes, semi-crystalline polymers, lipophilic gelling agents, and mixtures thereof.

14. The kit according to claim 1, wherein the first composition comprises at least one film-forming polymer.

15. The kit according to claim 1, wherein the second composition has a surface energy such that the difference between this surface energy and that of the first composition is greater than or equal to 7 mN/m.

16. The kit according to claim 1, wherein the second composition comprises an aqueous continuous phase.

17. The kit according to claim 1, wherein the second composition is free of wax.

18. The kit according to claim 1, wherein the second composition is free of surfactant.

19. The kit according to claim 1, wherein the second composition comprises at least one film-forming polymer.

20. The kit according to claim 1, wherein the second composition comprises a filler.

21. The kit according to claim 20, wherein the filler is a low-density filler.

22. The kit according to claim 1, wherein the second composition comprises low-density files in a content of 0.1% to 50% by weight, relative to the total weight of the second composition.

23. The kit according to claim 1, wherein the first and second compositions are included in the same packaging article equipped with two separate applicators.

24. The kit according to claim 1, wherein the first and second compositions are separate from one another.

25. A process, comprising applying a first composition to all or part of a keratin fibre, forming a first deposit, and applying a second composition onto all or part of the first deposit and/or of the keratin fibres, wherein the first and second compositions are as defined in claim 1.

26. The process according to claim 25, wherein said process forms pearls or bridges at the end of said keratin fibres on said keratin fibres.
